(19) United States
(12) Patent Application Publication
(10) Pub. No.: US 2025/0176821 A1
(43) Pub. Date: Jun. 5, 2025

(54) DISPLAY SYSTEM FOR MANUALLY GUIDED VISUAL EXAMINATION

(71) Applicant: Welch Allyn, Inc., Skaneateles Falls, NY (US)

(72) Inventors: David Gregory Perkins, Tully, NY (US); Joshua F. Hess, Moravia, NY (US); Lei Guo, Manlius, NY (US); David Villarreal, Syracuse, NY (US); Danielle Endres, Syracuse, NY (US)

(21) Appl. No.: 18/959,333

(22) Filed: Nov. 25, 2024

Related U.S. Application Data
(60) Provisional application No. 63/605,266, filed on Dec. 1, 2023.

(51) Int. Cl. A61B 3/00; A61B 3/14

(52) U.S. Cl. CPC A61B 3/005; A61B 3/14

(57) ABSTRACT

An example system is described herein for a display device to couple with a handheld diagnostic tool allowing an optical device to be converted to digitally imaging and back and used for performing a medical examination by a clinician. The display device includes an interface that includes the ability to attach to an eyepiece of the diagnostic device. The display device includes a camera within the channel to align with the eyepiece and capture image data through the diagnostic device when the display device is coupled with the diagnostic device. The display device has the camera essentially centered along a length and width of the body such that the display device maintains the balance of the diagnostic device. The display device further includes sensors to determine an orientation of the display device to annotate image data with an indication of a reference frame.

### CROSS REFERENCE TO RELATED APPLICATIONS

This application claims the benefit of U.S. Provisional Patent Application No. 63/605,266, filed Dec. 1, 2024, the entirety of which is incorporated by reference herein in its entirety for all purposes.

### TECHNICAL FIELD

This application relates generally to diagnostic systems and more specifically to an improved physical assessment device, which is configured for performing diagnostic patient examinations.

### BACKGROUND

In various medical fields, clinicians diagnose health and diseases of patients by operating vision aid instruments to examine parts of a patient's body. By way of example, during eye examinations by clinicians, ophthalmoscopes allow a clinician to examine the fundus, the rear interior of the eye, directly. During ear examinations by clinicians, otoscopes allow a clinician to examine the ear canal and eardrum. During examination of skin lesions, dermatoscopes allow a clinician to examine the surface and subsurface of the skin. Ophthalmoscopes, otoscopes, dermatoscopes, and other such vision aid instruments are compact, portable optical instruments which can be handheld or otherwise manually guided. An ophthalmoscope includes a lens which is held by a clinician, whether gripped directly or gripped by an attached member or device, between the patient and the clinician. The ophthalmoscope lens gathers or condenses light exiting a patient's pupil, presenting an image which the clinician can view.

The use of ophthalmoscopes, as well as other such manually guided vision aid instruments, presents substantial physical dexterity challenges, as the clinician must manually align the lens with the small opening provided by the pupil of the human eye, which is subject to constant, involuntary micro-movements. The manual guidance of such vision aid instruments, whether by a handle or by an attached member device of varying ease of handling, constantly tests the handling ability of clinicians. Likewise, in general-purpose applications for instrument-based visual examination of objects such as telescopy and microscopy, vision aids can be placed between an eyepiece of an optical instrument and an operator to facilitate viewing of images. Ease of handling can also facilitate the use of vision aids in these instrument-based applications.

Thus, there is a need to allow manually guided vision aids to be used more conveniently and in additional ways during and after clinical examinations to enable saving, sharing, and annotating of image data.

### SUMMARY

Various implementations of the present disclosure relate to a system for implementing a digital display device and image capture with a handheld diagnostic tool. The system includes a handheld diagnostic tool that includes a housing defining an optical port for performing a manually guided vision exam of a patient and an eyepiece optically connected to the optical port. The system also includes a device that has a body defining a channel on a first side of the housing, a camera positioned within the channel, and a display on a second side of the body. The channel is configured to releasably secure with the eyepiece to align the camera with the optical port and enable rotation of the device relative to the handheld diagnostic tool for reasons such as improved ergonomics while maintaining alignment of the camera with the optical port.

One general aspect includes a display device for a handheld diagnostic tool. The display device includes a body with an interface adjacent a surface of the body, the interface defining a channel extending along a first direction from an edge of the display device to a center portion of the display device. The interface includes a receiving portion adjacent the edge of the display device and defining an opening having a first width to receive an eyepiece of the handheld diagnostic tool. The interface also includes a middle portion positioned between the edge of the display device and the center portion along the first direction, the middle portion having a second width at the surface of the body and a third width at a depth below the surface of the body, where the second width is less than the third width. The interface further includes a locking portion positioned adjacent the center portion of the display device that includes one or more features to interface with the eyepiece to frictionally retain the eyepiece in a position. The device also includes a camera positioned within the channel adjacent the center portion of the body. The device also includes a display communicably coupled to the camera and configured to display image data captured by the camera.

One general aspect includes a device. The device has a body defining a channel on a first side. The device also includes a camera positioned within the channel. The device also includes a display on a second side of the body, where the channel is configured to releasably secure with an eyepiece of a handheld diagnostic tool to align the camera with an optical port of the handheld diagnostic tool and enable rotation of the device relative to the handheld diagnostic tool while maintaining alignment of the camera with the optical port.

### DESCRIPTION OF THE FIGURES

The following figures, which form a part of this disclosure, are illustrative of described technology and are not meant to limit the scope of the claims in any manner.

FIGS. 1A and 1B illustrate a handheld diagnostic tool with a display device capable of locking to and rotating relative to the handheld diagnostic tool, according to at least one example.

FIGS. 2A and 2B illustrate a device having a centrally located camera within a docking interface and a display for use with a handheld diagnostic tool, according to at least one example.

FIGS. 3A-3C illustrate various handheld diagnostic tools with display devices, according to at least some examples.

FIG. 4 illustrates a handheld diagnostic tool and a display device with a docking interface to couple with the handheld diagnostic tool, according to at least one example.

FIG. 5 illustrates a cross-sectional view of an eyepiece of a handheld diagnostic tool within a docking interface of a display device, according to at least one example.

FIG. 6 illustrates a system architecture and components of a device for capturing and displaying images through a handheld diagnostic tool, according to at least one example.

### DETAILED DESCRIPTION

Various implementations of the present disclosure will be described in detail with reference to the drawings, wherein like reference numerals present like parts and assemblies throughout the several views. Additionally, any samples set forth in this specification are not intended to be limiting and merely set forth some of the many possible implementations.

The following description provides for a device that provides a portable, battery-powered camera and/or display device to enable image capture (e.g., video or still image) through one or more different handheld diagnostic tools. In this manner, the description herein provides for digital imaging solutions to accompany conventional handheld diagnostic tools. The description herein provides for a removable attachment to various handheld diagnostic tools, determining and recording a reference frame to associate with captured images, enable rotation with respect to the handheld diagnostic tools (as well as removal), and also provides for ease of use by providing a balanced handheld device when coupled with the diagnostic tools to enable manipulation by a user.

The systems and devices described herein provide for the use of previously optical-only devices to interact in a manner beyond simple viewing. The systems and devices enable the user to save, share, annotate, and interact with stored images captured during an examination. The systems and devices described herein provide for digital image capture without requiring replacement of an entire suite of optical tools for examinations, such digital tools are much more expensive than optical viewing devices and the systems and devices described herein provide the benefits of the digital systems without the large cost of a suite of new digital tools.

The system and devices described herein provide improvements over existing designs that lack the secure attachment of the present systems and devices and also enable flexibility for various orientations and rotations as desired by a user. Accordingly, as the handheld diagnostic tools move during an exam, the systems and devices herein enable secure rotation to directly face a clinician while still recording and referencing a fixed reference frame. Existing solutions that implement a smartphone or other such device may have a camera positioned significantly off-center on the display device, which results in an awkward balance for a user trying to manipulate the tool. The systems and devices herein provide for centering the camera on a display device to improve balance and ergonomics when attached to a diagnostic tool such as an ophthalmoscope or otoscope (among other examples).

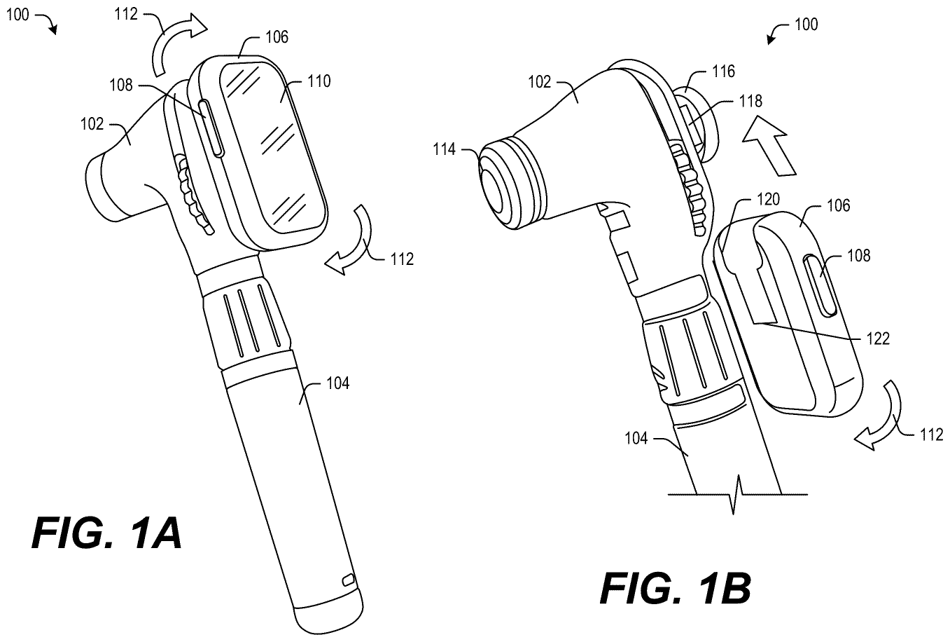

FIGS. 1A and 1B illustrate a handheld diagnostic tool 100 with a display device 106 capable of locking to and rotating relative to the handheld diagnostic tool 100, according to at least one example. Apparatuses discussed herein are directed to enabling digital image capture in connection with manual diagnostic tools. The camera of the systems and devices described herein provide a lens to implement the device for visual examination. In some examples, the handheld diagnostic tool 100 includes a lens, therefore the camera may, in some examples, not require a separate lens.

The handheld diagnostic tool 100 can be any instrument where a viewing lens system (not illustrated) is set internally within a head 102, which may or may not be further attached to a handle 104. Depending on the nature of the instrument, the handheld diagnostic tool 100 can include multiple viewing lenses having various diopter values among various ranges of diopter values or with a continuously variable focusing method. Diopter values describe the optical power of a lens to converge light, thereby compensating for refractive error in an eye of a patient (which can result in light exiting the patient's pupil converging on an unclear image), or refractive error in an eye of a clinician (which can result in the clinician failing to clearly see an image converged from an eye of the patient).

By way of example, in the event that the handheld diagnostic tool 100 is an ophthalmoscope, it can include multiple viewing lenses having respective diopter values ranging from −20 diopter to 25 diopters (and can further include additional lens having negative diopter values, without limitation). In the event that the handheld diagnostic tool 100 is an otoscope, a dermatoscope, and other similar instruments, it can likewise include one or more viewing lenses; rather than diopter values, viewing lenses of an otoscope or a dermatoscope generally have focal length specified. In some examples, the dermatoscopes and/or otoscopes may include lenses with variable focal length to enable focusing. A viewing lens of an otoscope may have a focal length in a range of 10 millimeters to approximately 50 mm, and a viewing lens of a dermatoscope can have a focal length in a range of 10 millimeters to approximately 50 millimeters, by way of example.

It should be understood that, while a viewing lens according to example embodiments of the present disclosure can magnify images, magnification of a viewing lens is not relevant to understanding the present description. Thus, by way of example, a viewing lens according to example embodiments of the present disclosure can have angular magnification of approximately 1×, or can have any other arbitrary magnification.

A viewing lens can be set within the head 102 of the handheld diagnostic tool 100 that transports light that enters through a patient end 114 to the eyepiece 116. Furthermore, in some examples, aspheric lenses found in ophthalmoscopes can have a diopter value of 20 diopters, 28 diopters, 60 diopters, 78 diopters, 90 diopters, and the like; and any of these aspheric lenses can be set within the handheld diagnostic tool 100, increasing optical power to converge light through one or more viewing lenses.

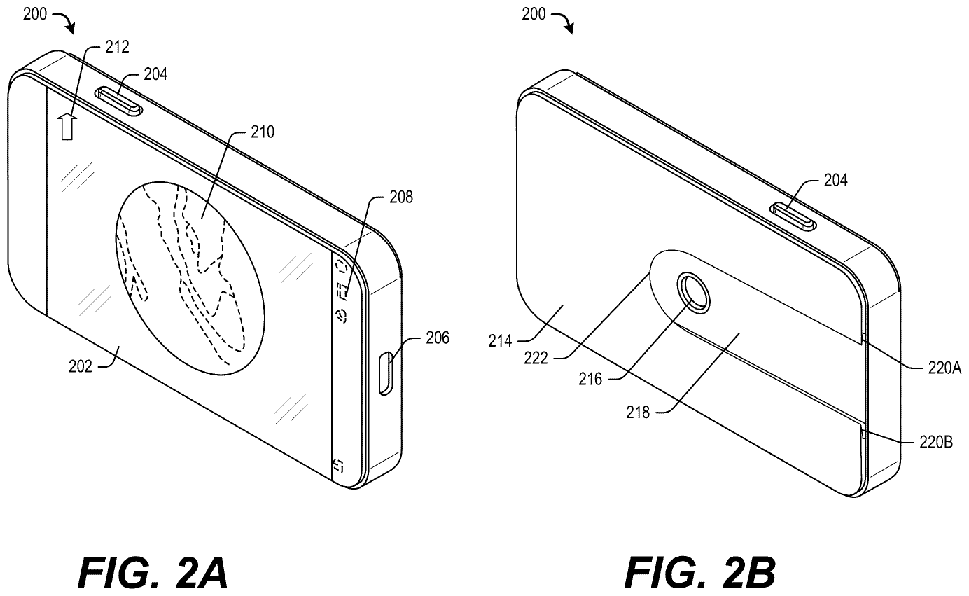

Thus, as illustrated in FIGS. 1A-1B, a display device 106 can be coupled to a handheld diagnostic tool 100 such that a prime lens and an aperture of a camera positioned on the back of the display device 106 (see e.g., FIGS. 2A-2B) is aligned with a viewing lens (e.g., within eyepiece 116). By interposing this viewing lens in an optical axis of the camera, where the optical axis describes a path by which light is acquired by an image sensor of the camera after passing through the prime lens and the aperture, the viewing lens converges light into images which can be captured by the camera, even if the light originates from beyond a native focal length range of the camera. In the event that the viewing lens has a diopter value comparable to an lens system found in fundus cameras, the viewing lens can configure the image sensor of the camera to capture images of the rear interior of the human eye by acquiring light on an optical path through the pupil of the human eye while focusing on the fundus of the human eye.

It should be understood that a handheld diagnostic tool 100 such as an ophthalmoscope having multiple viewing lenses can be configured to mechanically swap between the multiple viewing lenses or otherwise adjust the focus, to vary a focal length which is interposed in the optical axis.

A person who is accustomed to handling any kind of handheld diagnostic tool 100 to visually examine subjects through a lens can, in a similar manner, handle any kind of handheld diagnostic tool 100 having a display device 106 coupled thereto (whether the device includes a handle 104 or not; in the event that the visual aid does not include a handle, the person can handle the handheld diagnostic tool 100 by handling the display device 106) in the course of visually examining subjects, to concurrently operate a camera of the display device 106 to capture images of an eye (in the event that the handheld diagnostic tool 100 is an ophthalmoscope); capture images of an ear (in the event that the handheld diagnostic tool 100 is an otoscope); capture images of skin (in the event that the handheld diagnostic tool 100 is a dermatoscope); capture images through an optical instrument (such as a microscope, telescope, or borescope); or capture images of any other subject being examined.

The display device 106 includes a button 108 for interacting with the display device 106, for example to turn the display device 106 on and/or off, capture images, and other such operations. The display device 106 also includes a screen 110 for displaying image data and/or interacting with the display device 106. In some examples, the display device 106 may include multiple buttons and/or a touchscreen for the button 108 and/or screen 110 to provide various avenues for a clinician to interact with the display device 106 and/or images captured thereby. In some examples, the clinician may use the button 108 and/or screen 110 to perform operations such as saving an image, providing an annotation (e.g., by dictating or otherwise inputting a note), transmitting the image to an additional device, altering a setting for the camera, and other such operations.

The display device 106 includes a camera positioned at or near a center of the display device 106. The camera is positioned within the channel 120 such that the eyepiece 116, and particularly a viewing axis or port of the eyepiece 116 is aligned with the camera regardless of the orientation of the display device 106 relative to the handheld diagnostic tool 100. Centering the camera within the body of the display device 106 provides for even balance of the handheld diagnostic tool 100 during use, regardless of the orientation. In this manner, the clinician, who may be trained to use the handheld diagnostic tool 100 without a display device 106, may perform examinations without hindrance or awkwardness that may be caused by using a device such as a smartphone that has an off-center camera.

The display device 106 may rotate around the eyepiece 116 (e.g., in a direction 112) while maintaining an alignment of the camera with the eyepiece 116. In this manner, a clinician may rotate the display device 106 while performing an examination to reorient the screen 110 in a preferred orientation. In some examples, the display device 106 may be rotated relative to the head 102 by simply twisting the display device 106 to the preferred orientation. Features within the channel 120 may engage with detents or other features of the eyepiece 116 to provide positive engagement and/or locking and/or resistance to movement through a frictional interface or otherwise such that the display device 106 remains in the preferred orientation after being positioned by the clinician. In some examples, the display device 106 may couple to the head 102 in four distinct orientations (e.g., evenly spaced at angles of 90 degrees). The display device 106 may be removed from the head 102 to turn 90 degrees, 180 degree, or 270 degrees and then reattached to the head 102 in the altered orientation.

The display device 106 couples to the handheld diagnostic tool 100 through the use of a channel 120 on a back of the display device 106 that receives the eyepiece 116 and an eyepiece support 118 of the handheld diagnostic tool 100. The channel 120 may receive the eyepiece 116, which has a width or diameter that is greater than the eyepiece support 118 and capture, at least partially, the eyepiece 116 within the channel 120. In particular, the channel 120 may extend to a center portion of the display device 106 to an end 122 that contacts the eyepiece support 118 to “bottom out” or provide a positive stop to ensure proper positioning and alignment of the eyepiece with a camera positioned within the channel 120 near the end 122.

In some examples, cases, due to the shape of the display device 106 (e.g., rectangular) and when used in cases where the maneuvering space is limited, such as an ear exam, users may prefer to reorient the display. In some cases, the user may rotate the handheld diagnostic tool 100 off to the side and so prefer to turn the display device 106 to orient it more towards their eye and/or in an orientation that they prefer. Also, in some cases, the display device 106 may be locked to the instrument head to reduce the chance of theft or the display device 106 dislodging from the head 102 during use.

The display device 106 may couple to the head 102 in four or more orientations. In some examples, a nearly infinite number of angles may be possible by providing a frictional engagement with the eyepiece 116 and rotating the display device about the eyepiece 116. In some examples, the display device 106 may couple at distinct angles, such as may be defined by one or more flat sides of the eyepiece support 118 or other features of the head 102 as they interact with the channel 120 and/or end 122.

In some examples, the display device 106 may couple to the handheld diagnostic tool at a range of positions and angles, including at angles and positions away from perpendicular angles. Additionally, the display device 106 may couple to the head 102 through an interface that locks, and may be released by interacting with the button 108 or other such features, to deter theft and/or removal by unauthorized parties. Such a locking and/or retention mechanism may use frictional engagement to retain the display device 106 in position and may also provide a physical block to prevent or resist removal, either intentional or accidental.

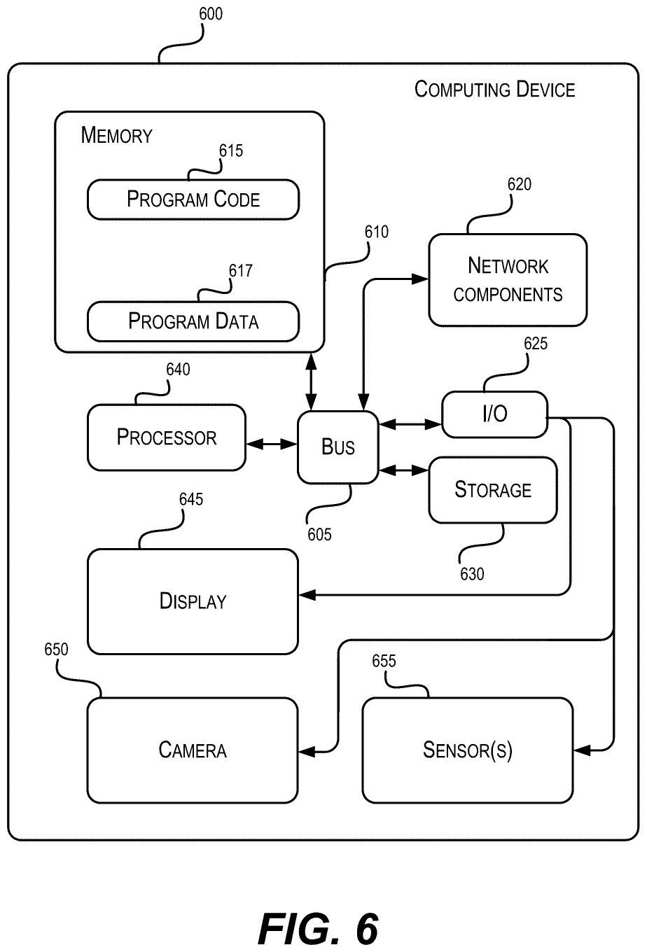

The display device 106 may include components such as shown and described in FIG. 6 that enable one or more additional operations in addition to image capture. For example, the display device 106 may be capable of detecting an orientation of the display device 106 and annotating the image data and/or providing metadata to accompany the image data indicative of the orientation of the display device 106. In some examples, anatomy or portions of the body that may be imaged may be difficult to orient based on the image data alone. In some examples, the display device 106 may determine an orientation with respect to a gravitational reference frame (e.g., with respect to gravity) or through other means, such as image analysis, such that the user may turn and manipulate the handheld diagnostic tool 100 as they wish during an examination and the orientation of the patient (e.g., direction for up/down) may be annotated for later reference. The display device 106 may additionally annotate and/or record data with respect to the relative orientation of the display device 106 relative to the handheld diagnostic tool 100. In some examples, the orientation and/or a gravitational reference (orientation information) may be displayed on the screen 110 and/or associated with the image data as image data is captured.

In some examples, the display device 106 may be capable of processing image data from the camera to determine an orientation of the display device 106 relative to the patient and/or to a gravitational reference frame. For instance, the display device 106 may include processing components, such as shown and described with respect to FIG. 6 that may be used to perform image recognition techniques using artificial intelligence and/or algorithms to determine anatomical components visible within the image data, and based on reference image data and reference orientation information, determine an orientation for the camera. For example, the processing components may include machine learned algorithm or artificial intelligence system trained using image data of an ear canal of a patient along with annotations indicating orientations of the image data (e.g., gravitational reference or UP/DOWN reference). The machine learned algorithm or artificial intelligence system may then analyze the image data captured by the camera of the display device 106 and determine an orientation to associate with the image data.

The display device 106 may additionally be used to record, annotate, edit, or otherwise interact with information such as patient information, timestamps, details of the system (e.g., what brand or model of handheld diagnostic tool 100 and/or display device 106 and/or serial number of connected devices), or other such information. The data recorded by the display device 106 may be stored and may be annotated onto the image data with a watermark, associated as metadata, or otherwise accompany and/or be associated with the captured images. The display device 106 may further be capable of transmitting information to one or more additional devices or systems such that the display device 106 can transmit to a computer for further analysis and display, for recording to an electronic medical record of a patient, or other such use cases.

FIGS. 2A and 2B illustrate a device 200 having a centrally located camera 216 within a locking interface 218 and a display 202 for use with a handheld diagnostic tool, according to at least one example. The device 200 may be an example of a display device 106, such as shown and described with respect to FIGS. 1A-1B. The device 200 may include components such as those shown and described with respect to FIG. 6 below. The device 200 includes a display 202 where a user may interact with and/or view the image data 210 captured by the camera 216. The display 202 may be rectangular, square, or any other shape. The display 202 may include a representation of the image data 210 captured by the camera 216. The display 202 may also include information 208 such as a status of a battery and/or connection status of the device 200 with one or more other systems. In some examples, the information 208 may include information such as an identifier of the device 200, level of charge of a battery, identifier or indication of a handheld diagnostic tool that is connected to the device 200, or other such information.

The device 200 includes sensors, such as shown and described in FIG. 6 and including an orientation sensor that may be used to determine an orientation of the device 200 with respect to a gravitational, or other, reference frame. The sensors may determine a direction of gravitational acceleration, in some examples the sensors may include an accelerometer, gyroscope, magnetometer, and/or other such components such that the device 200 is capable of determining an orientation with respect to a reference frame and to determine a change in orientation over time as the device 200 is manipulated by a user. The device 200 may determine the orientation using the sensor data and display an indicator 212 on the display 202 showing a representation of the orientation of the device on the image data 210. The representation may be added to the image data as a watermark, metadata, and other such data associated with the image data 210.

The display 202 may include a screen that displays color and/or grayscale or other such representations of image data. In some examples, the display 202 may include a touchscreen that enables interactions with the image data 210 through touch inputs on the display 202. The display 202 may have a bezel in some examples, or may extend across the extent of the body without a bezel, thereby providing as large of a viewing display as possible. The display 202 may have a resolution of at least two hundred and fifty pixels per inch to provide sufficient clarity and detail for a user to view the image data 210. The display 202 may have a diagonal dimension in a range of three to six inches, or more or less in some examples. The display 202 may have any shape or aspect ratio, for example including 16:9, 4:3, 1:1, round, or other such shapes.

The device 200 includes a button 204 that may be used to interact with the device 200 such as to power on the device, capture a still image, lock/unlock the locking interface 218, and/or other such interactions. The device 200 also includes a port 206 that may receive a charging cable and/or provide an input/output for a speaker and/or microphone.

The locking interface 218 is positioned on a surface 214 of the device 200 opposite from the display 202. The locking interface 218 may include a slot that clips onto the head of the handheld diagnostic tool to selectively prevent removal (e.g., until the lock is disengaged). The locking interface 218 may couple with an eyepiece of a handheld diagnostic tool and align the camera 216 with a viewing port of the handheld diagnostic tool. The camera 216 is positioned within a channel defined by a first edge 220A and a second edge 220B such that the eyepiece is aligned with the camera 216 regardless of the orientation of the device 200 relative to the handheld diagnostic tool. The camera 216 may be positioned vertically and horizontally centered (e.g., at a center of the surface 214) of the device 200. Centering the camera 216 on the surface 214 provides for even balance of the handheld diagnostic tool during use, regardless of the orientation as the handheld diagnostic tool will have a similar balance if the device 200 is oriented in varying positions.

The channel defined by the first edge 220A and the second edge 220B may partially enclose and/or surround the eyepiece of the handheld diagnostic tool, such as the eyepiece 116. The first edge 220A and the second edge 220B extend from an edge of the device 200 towards the center of the surface 214. The first edge 220A and the second edge 220B define a width of the channel, the width being smaller at the surface 214 than adjacent the camera 216. The first edge 220A and second edge 220B form overhanging edges for retaining the eyepiece against the device. At the edge of the device, the first edge 220A and the second edge 220B form an opening sufficiently wide to accommodate the widest portion of the eyepiece. The first edge 220A and the second edge 220B may have a profile, along the height of the channel, which corresponds to the outer profile of the eyepiece such that the eyepiece contacts the inner surface of the first edge 220A and the second edge 220B to provide a frictional interface to retain the eyepiece in position relative to the device 200.

A third edge 222 of the locking interface 218 provides for a physical stop to resist further movement of the eyepiece along the channel, and thereby ensure alignment (along the direction of the channel) of the eyepiece with the camera 216. The first edge 220A and the second edge 220B are positioned with the camera 216 centered in-between to provide alignment of the camera 216 in a direction perpendicular to a length of the channel, with the eyepiece. The third edge 222 may be rounded, as shown in FIGS. 2A-2B and/or may have a straight or flat profile, such as depicted in FIGS. 1A-1B. The rounded profile may enable or allow rotation of the device 200 relative to a head of the handheld diagnostic tool while the eyepiece is at the end of the channel adjacent the camera 216 (e.g., without having to remove the eyepiece from the channel).

The device 200 may rotate around the eyepiece while maintaining an alignment of the camera 216 with the eyepiece. In this manner, a clinician may rotate the device 200 while performing an examination to reorient the display 202 in a preferred orientation. In some examples, the device 200 may be rotated relative to the head of the handheld diagnostic tool by simply twisting the device 200 to the preferred orientation. Features within the channel, such as indentations, may engage with detents or other features of the eyepiece to provide positive engagement, registration (e.g., known angle positioning), and/or locking, and/or resistance to movement through a frictional interface or otherwise such that the device 200 remains in the preferred orientation after being positioned by the clinician. In some examples, the device 200 may couple to the head of the handheld diagnostic tool in four distinct orientations (e.g., evenly spaced at angles of 90 degrees). The device 200 may be removed from the head to turn 90 degrees, 180 degrees, or 270 degrees and then reattached to the head in the new orientation.

The device 200 couples to the handheld diagnostic tool through channel on the surface 214 as described above. The channel may receive the eyepiece, which has a width or diameter that is greater than the eyepiece support and capture, at least partially, the eyepiece within the channel between the first edge 220A and the second edge 220B. In particular, the channel may extend to a center portion of the device 200 to an end that contacts the eyepiece support to “bottom out” or provide a positive stop to ensure proper positioning and alignment of the eyepiece with a camera positioned within the channel near the end.

In some examples, cases, due to the shape of the device 200 (e.g., rectangular) and when used in cases where the maneuvering space is limited, such as an ear exam, users may prefer to reorient the display 202. In some cases, the user may rotate the handheld diagnostic tool off to the side and so prefer to turn the device 200 to orient it more towards their eye and/or in an orientation that they prefer. Also, in some cases, users may prefer that the device 200 be locked to the instrument head to reduce the chance of theft or the device 200 dislodging from the head during use.

The device 200 may couple to the head in four or more orientations. In some examples, a nearly infinite number of angles may be possible by providing a frictional engagement between the eyepiece and the channel defined by the first edge 220A, second edge 220B, and the third edge 222. In some examples, the device 200 may couple at distinct angles, such as may be defined by one or more flat sides of the eyepiece support or other features of the head as they interact with the channel, such as shown and described with respect to FIGS. 1A-1B.

In some examples, the device 200 may couple to the handheld diagnostic tool at a range of positions and angles, including at angles and positions away from perpendicular angles. Additionally, the device 200 may couple to the head through an interface that locks, and may be released by interacting with the button 204 or other such features, to deter theft and/or removal by unauthorized parties. Such a locking and/or retention mechanism may use frictional engagement to retain the device 200 in position and may also provide a physical block to prevent or resist removal, either intentional or accidental.

The device 200 may be compact and lightweight, having a thickness that may be less than one inch or approximately twenty-five millimeters in some examples, for instance from the display 202 to the surface 214. In some examples, the thickness may be in a range of one-quarter inch to three-quarters of an inch. The device 200 may have a width and a height each in a range of one to five inches, or more. The device 200 may have an overall weight of less than two hundred and fifty grams in some examples, such that use of the device 200 with a handheld diagnostic tool is not impeded or made more difficult due to the added weight. In some examples, the device 200 may have an overall weight of less than one hundred grams. The limited weight of the device may enable the user to easily manipulate the handheld diagnostic tool with the device 200 attached.

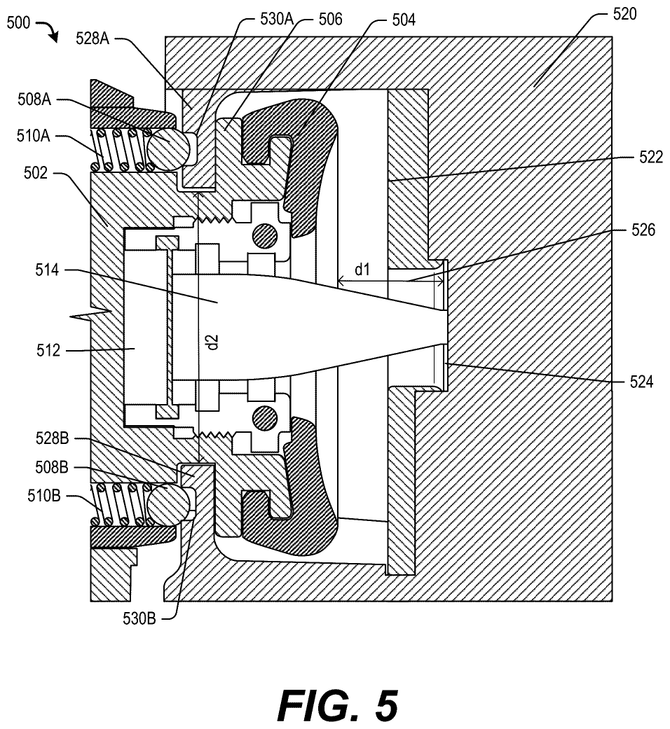

The device 200 may capture image data as still images and/or video data. In some examples, the user may use the display to capture video at up to and in excess of 4K resolution. The device 200 may store the image data on-board the device 200 and/or may communicate the image data to an external storage device. The camera 216 is centered on the body of the device 200 (e.g., centered on the surface 214 at the rear of the device 200). The camera 216 may include a color image sensor and may be a 12-megapixel image sensor or other suitable image sensor. The camera 216 may be situated within the channel of the locking interface 218 such that the camera 216 does not protrude out the surface 214. The camera 216 may include an autofocus camera module and/or camera module that may be manually focuses by a user. In some examples, the camera 216 may have a wide angle and telephoto mode. The camera 216 may have a field of view in a range of twenty-two degrees to twenty-eight degrees. In some examples, the camera 216 may have two modes, such as the wide and telephoto modes that provide a field of view of around twenty degrees and around forty degrees. The camera 216 may also provide digital and/or optical zoom capability. The camera 216 may include telephoto zoom to compensate for the field of view of the handheld diagnostic tool (e.g., to compensate for the limited zoom capability). The camera 216, within the channel, may have a spacing of a fixed distance from the eyepiece. For example, as shown and described with respect to FIG. 5, the distance from the eyepiece to the lens and/or sensor of the camera may be a fixed distance such as in a range of two to fifteen millimeters. The distance from the image sensor to the entrance of the eyepiece may be based on the field of view of the image sensor and are positioned such that the image sensor views the image from the eyepiece without clipping of the edges or corners of the image data.

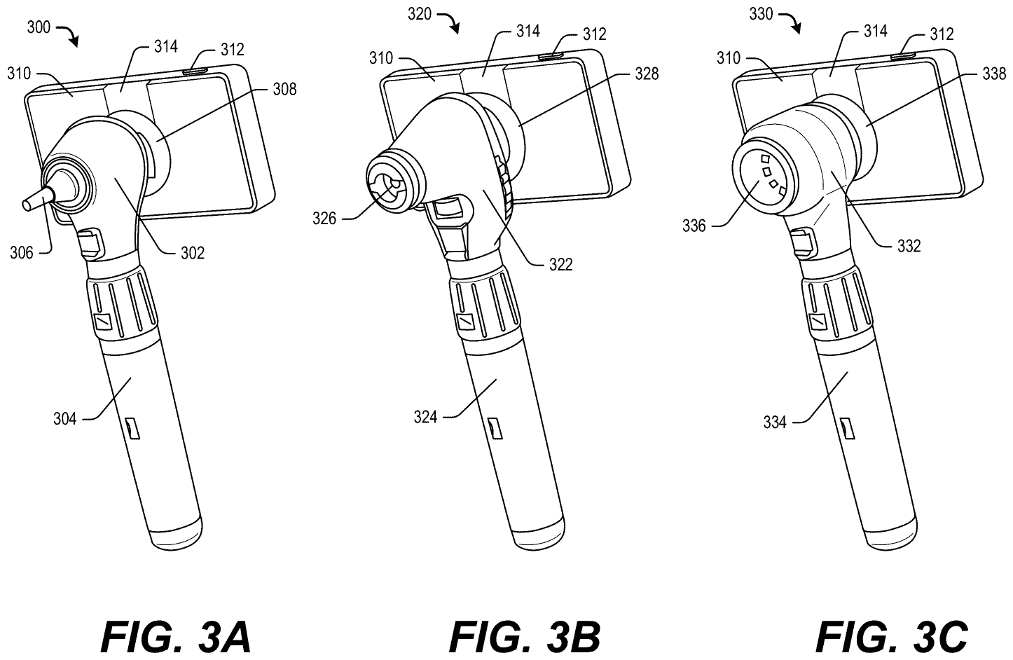

FIGS. 3A-3C illustrate various handheld diagnostic tools with display devices, according to at least some examples. The handheld diagnostic tools depicted in FIGS. 3A-3C provide for digital imaging while using tools that meant for optical viewing by a caregiver. The devices and systems described herein provide for an imaging device that may be used across handheld diagnostic tools, and may be swapped back and forth in some examples, thereby reducing cost for a caregiver facility while also enabling digital imaging and storage of digital images during an examination.

FIG. 3A illustrates an otoscope 300 for diagnosing conditions of the ear of a patient. The otoscope 300 is designed primarily for performing diagnostic examinations of the ear of a patient, although the herein described physical assessment device can also be used for examining other anatomical cavities (i.e., the nose, throat) of a patient. The otoscope 300 is defined by an head 302 that is releasably attached to the upper end of an instrument handle or handle portion 304. The instrument handle portion 304 is sized and shaped to permit the otoscope 300 to be handheld and is further configured to retain at least one battery (not shown in these views) for powering a light source (not shown) contained in the head 302. The contained light source is energized by an on-off button or rheostat disposed on the exterior of the handle portion 304, wherein the illumination output of the contained light source can be controlled using a rheostat, the latter including a twistable portion formed on the handle portion 304. The contained battery can preferably be recharged via a charging port, which is provided in the bottom end of the handle portion 304.

The head 302 includes a body or housing having a distal or patient end 306 and an opposing proximal or caregiver end including an eyepiece 308. A hollow speculum tip element is releasably attached to the patient end 306 of the head 302, the speculum tip element being designed and shaped to fit a predetermined distance into the ear canal while the eyepiece 308 of the head 302 includes an adapter interface to receive the display device 310.

The display device 310 includes a locking interface 314, such as shown and described with respect to FIGS. 1A-1B and 2A-2B, that interfaces with the eyepiece 308, and specifically with the adapter interface to secure the display device 310 to the otoscope 300. The display device 310 further includes a button 312 that may be used to release the eyepiece 308 from the locking interface 314, wake the display device 310, cause the display device 310 to enter a sleep mode, capture video and/or image data, and or otherwise interact with the display device 310. The display device 310 may be rotated with respect to the otoscope 300 such that the display device 310 may be oriented substantially vertically or horizontally, or at any angle desired by a caregiver.

FIG. 3B illustrates an ophthalmoscope 320 for diagnosing conditions associated with the eye of a patient. The ophthalmoscope 320 is designed primarily for performing diagnostic examinations of the eye of a patient, although the herein described physical assessment device can also be used for examining other anatomical features of a patient. The ophthalmoscope 320 is defined by an head 322 that is releasably attached to the upper end of an instrument handle or handle portion 324. The instrument handle portion 324 is sized and shaped to permit the ophthalmoscope 320 to be handheld and is further configured to retain at least one battery or wall system (not shown in these views) for powering a light source (not shown) contained in the head 322. The contained light source is energized by turning the rheostate disposed on the exterior of the handle portion 324, wherein the illumination output of the contained light source can be controlled using a rheostat, the latter including a twistable portion formed on the handle portion 324. The contained battery can preferably be recharged via a charging port, which is provided in the bottom end of the handle portion 324.

The head 322 includes a body or housing having a distal or patient end 326 and an opposing proximal or caregiver end including an eyepiece 328. An imaging component of the patient end 326 enables imaging and viewing of the eye of the patient from the eyepiece 328. The eyepiece 328 of the head 322 includes an adapter interface to receive the display device 310.

FIG. 3C illustrates a dermatoscope 330 for diagnosing conditions associated with the skin of a patient. The dermatoscope 330 is designed primarily for performing diagnostic examinations of the skin of a patient, although the herein described physical assessment device can also be used for examining other anatomical features of a patient. The dermatoscope 330 is defined by a head 332 that is releasably attached to the upper end of an instrument handle or handle portion 334. The instrument handle portion 334 is sized and shaped to permit the dermatoscope 330 to be handheld and is further configured to retain at least one battery (not shown in these views) for powering a light source (not shown) contained in the head 332. The contained light source is energized by an on-off button disposed on the exterior of the handle portion 334, wherein the illumination output of the contained light source can be controlled using a rheostat, the latter including a twistable portion formed on the handle portion 334. The contained battery can preferably be recharged via a charging port, which is provided in the bottom end of the handle portion 334.

The head 332 includes a body or housing having a distal or patient end 336 and an opposing proximal or caregiver end including an eyepiece 338. An imaging component of the patient end 336 enables imaging and viewing of the skin of the patient from the eyepiece 338. The eyepiece 338 of the head 332 includes an adapter interface to receive the display device 310.

The display device 310 may be moved between different devices, such as from the otoscope 300 to the ophthalmoscope 320 to the dermatoscope 330. In some examples, the display device 310 may be capable or enabled with sensors to detect the type of handheld diagnostic tool it is connected to and may change one or more operations or information available on the display based on detecting the different devices. In an example, the display device 310 may include a sensor such as a capacitive sensor or physical coupling sensor to detect when the display device 310 is connected to a handheld device. In some examples, the sensor of the display device 310 may read a visual indicator of the handheld device to determine the type of handheld device the display device 310 is coupled to. The sensor of the display device 310 may also read, using a non-contact sensor, a chip, antenna, or other such contactless communication sensor, a chip that may be embedded or attached to the handheld device. The display device 310 may therefore alter the information on the display and/or associated with the image data (e.g., with a watermark or metadata) to indicate that the handheld device is a certain type of device.

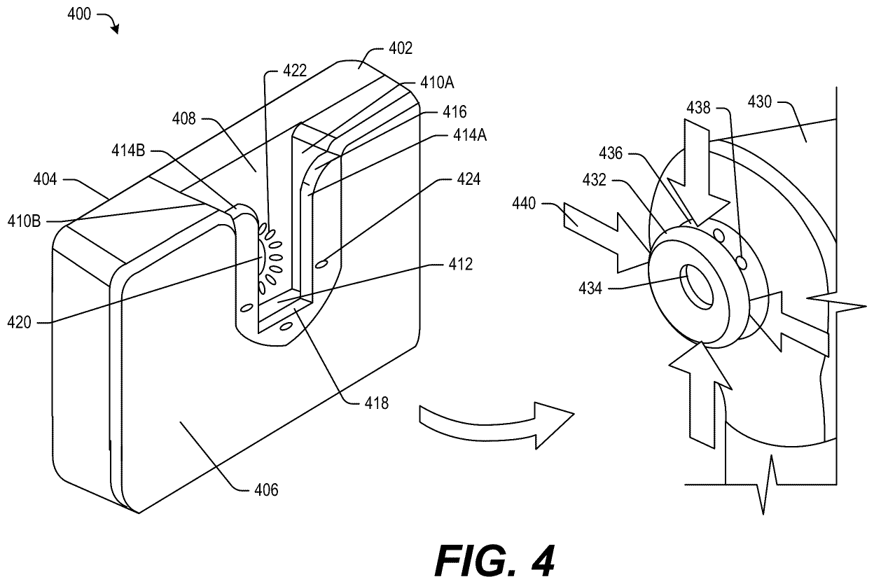

FIG. 4 illustrates a system 400 including a handheld diagnostic tool 430 and a display device 402 with a docking interface 416 to couple with the handheld diagnostic tool 430, according to at least one example. The display device 402 may be an example of the device 200 described with respect to FIGS. 2A-2B, or other such devices described herein. The display device 402 is equipped with a display 404 on a first side of the display device 402. The display device 402 has a second side 406 that includes the docking interface 416 for interfacing with the handheld diagnostic tool 430.

The docking interface 416 includes a channel 408 that extends from an edge of the display device 402 to a center portion (e.g., at or near a center of the second side 406). The docking interface 416 includes a first wall 410A, second wall 410B, and third wall 412 that are positioned between a bottom surface of the channel 408 and retaining wall 414A, retaining wall 414B, and retaining wall 418. The first wall 410A, second wall 410B, and third wall 412 define an internal passage of the channel 408 that has a width, between the first wall 410A and the second wall 410B, that is sized to accept an eyepiece 432 of the handheld diagnostic tool 430. The retaining wall 414A, retaining wall 414B, and retaining wall 418 define an outer portion of the docking interface 416 that is at or adjacent the second side 406. The width between the retaining wall 414A and the retaining wall 414B is a second width that is less than a first width from the first wall 410A to the second wall 410B.

The display device 402 includes a camera 420 within the channel 408. The camera 420 is positioned adjacent an end of the channel 408 near and/or at the center of the display device 402 (e.g., the center across the width and length of the second side 406. The camera 420 is positioned such that the display device 402 may be rotated relative to the handheld diagnostic tool 430 while still maintaining alignment of the camera 420 and an optical passage 434 of the eyepiece 432.

The display device 402 may include one or more sensors 422 positioned around the camera 420 and/or around the channel 408, such as on a bottom of the channel 408, on the first wall 410A, second wall 410B, third wall 412, retaining wall 414A, retaining wall 414B, retaining wall 418, and/or second side 406. The one or more sensors 422 may be configured to determine when the display device 402 is coupled with a handheld diagnostic tool 430, determine a type of device, determine a unique identifier of the device, and other such information. The information gathered by the one or more sensors 422 may be used by the display device 402 to determine to wake the display device 402 (e.g., when detecting a new coupling), type of information to display alongside the image data (e.g., information specific to the type of device), and other such information. In some examples, the one or more sensors 422 may communicatively couple with the handheld diagnostic tool 430 to display information related to battery status, settings, preferences, and other information related to the use of the handheld diagnostic tool 430. The one or more sensors 422 may also include one or more sensors, as discussed herein, to determine an orientation of the display device 402, such as relative to a reference frame (e.g., gravity). The gravitational reference frame, or other such information determined based on data from the one or more sensors 422 may be displayed on the display 404 and/or stored in connection with image data captured by the camera 420.

The display device 402 includes recesses or cavities to receive one or more detents 438 positioned around the perimeter of the eyepiece 432. As illustrated with respect to FIG. 5, the detents 438 and the recesses 424 may be used to resist removal of the display device 402 from the handheld diagnostic tool 430 and/or to selectively interact to preferentially lock the display device 402 at particular rotational intervals with respect to the eyepiece 432.

The display device 402 may couple to the handheld diagnostic tool 430 by receiving the eyepiece in the channel 408 with the retaining wall 414A, retaining wall 414B, and retaining wall 418 resisting removal of the eyepiece 432 from the channel 408. The retaining wall 414A, retaining wall 414B, and retaining wall 418 may fit between the eyepiece 432 and a head of the handheld diagnostic tool 430, where an eyepiece support 436 may have a width and/or diameter that is less than the width or diameter of the eyepiece 432 and therefore fits within the width between the retaining wall 414A and the retaining wall 414B.

The display device 402 may couple to the head of the handheld diagnostic tool 430 in four or more orientations, such as indicated by directions 440. In some examples, a nearly infinite number of angles may be possible by providing a frictional engagement with the eyepiece 432 and the channel 408 and rotating the display device 402 about the eyepiece 432. In some examples, the display device 402 may couple at distinct angles, such as may be defined by one or more flat sides of the retaining walls, eyepiece support 436, or other features of the head and/or channel 408.

In some examples, the display device 402 may couple to the handheld diagnostic tool 430 at a range of positions and angles, including at angles and positions away from perpendicular angles. Additionally, the display device 402 may couple to the head through an interface that locks, and may be released by interacting with a button or other such features, to deter theft and/or removal by unauthorized parties. Such a locking and/or retention mechanism may use frictional engagement to retain the display device 402 in position and may also provide a physical block to prevent or resist removal, either intentional or accidental.

FIG. 5 illustrates a cross-sectional view of an eyepiece of a handheld diagnostic tool 500 within a docking interface of a display device 520, according to at least one example. The display device 520 includes an opening 522 that includes an image sensor 524 to capture image data through the eyepiece of the handheld diagnostic tool 500. The handheld diagnostic tool 500 may include some or all of the components described herein. The handheld diagnostic tool 500 includes a head 502 that has various optical elements for viewing a portion of a patient during an examination. The head 502 includes a brow rest 504 as part of the eyepiece as well as an eyepiece support 506 that extends from the head to the brow rest 504. A distance, d1, shown by element 526 from the brow rest 504 to the image sensor 524 may be fixed, and may be in a range of three to fifteen millimeters. The distance, d1, may allow light to travel through optical elements, such as an optical passage 512 of the head 502 and into the field of view of the image sensor 524, such as depicted by the optical field of view 514. The distance, d1, enables the image sensor to capture a full field of view through the handheld diagnostic tool 500.

The eyepiece support 506 has a diameter or width d2 such that it fits within a space formed between a first wall 528A and a second wall 528B. The width of the brow rest 504 prevents the brow rest 504 from being removed from the docking interface. The first wall 528A and the second wall 528B each have respective recesses 530A and 530B that face outwards away from the main body of the display device 520. The recesses 530A and 530B interface with detent balls 508A and 508B that are biased by springs 510A and 510B. The detent balls 508A and 508B may positively engage with the recesses 530A and 530B to provide an additional frictional interface to retain the display device 520 on the handheld diagnostic tool 500.

FIG. 6 illustrates a system architecture and components of a device for capturing and displaying images through a handheld diagnostic tool, according to at least one example. The computing device 600 of FIG. 6 may illustrate components of the display device discussed herein.

The computing device 600 can include a processor 640 interfaced with other hardware via a bus 605. A memory 610, which can include any suitable tangible (and non-transitory) computer readable medium, such as RAM, ROM, EEPROM, or the like, can embody program components (e.g., program code 615) that configure operation of the computing device 600. Memory 610 can store the program code 615, program data 617, or both. In some examples, the computing device 600 can include input/output (“I/O”) interface components 625 (e.g., for interfacing with a display 645, keyboard, mouse, and the like) and additional storage 630.

The computing device 600 executes program code 615 that configures the processor 640 to perform one or more of the operations described herein such as determination or the orientation of the display device and other such operations. The program code 615 may be resident in the memory 610 or any suitable computer-readable medium and may be executed by the processor 640 or any other suitable processor.

The computing device 600 may generate or receive program data 617 by virtue of executing the program code 615. For example, sensor data, usage counter, authenticated messages, trip flags, and other data described herein are all examples of program data 617 that may be used by the computing device 600 during execution of the program code 615.

The computing device 600 can include network components 620. Network components 620 can represent one or more of any components that facilitate a network connection. In some examples, the network components 620 can facilitate a wireless connection and include wireless interfaces such as IEEE 802.11, BLUETOOTH™, or radio interfaces for accessing cellular telephone networks (e.g., a transceiver/antenna for accessing CDMA, GSM, UMTS, or other mobile communications network). In other examples, the network components 620 can be wired and can include interfaces such as Ethernet, USB, or IEEE 1394.

Although FIG. 6 depicts a computing device 600 with a processor 640, the system can include any number of computing devices 600 and any number of processor 640. For example, multiple computing devices 600 or multiple processor 640 can be distributed over a wired or wireless network (e.g., a Wide Area Network, Local Area Network, or the Internet). The multiple computing devices 600 or multiple processor 640 can perform any of the steps of the present disclosure individually or in coordination with one another.

The computing device 600 also includes a camera 650 and one or more sensor(s) 655. The camera 650 may include one or more image capture devices included within the display device(s) discussed herein. The camera 650 is in communication, through the interface components 625, but may, in some examples capture image data to provide to the memory, storage, and/or one or more other components of the computing device 600 through other communication means. The computing device 600 also includes one or more sensor(s) 655 that may include an accelerometer, interlock sensor, hall effect sensor, interlock sensor, orientation sensor (e.g., with respect to a gravitational reference frame), or other such sensors. The sensor(s) 655 may capture data regarding the display device and/or the environment and communicate with one or more other components of the computing device 600 to perform the operations described herein.

While the present subject matter has been described in detail with respect to specific aspects thereof, it will be appreciated that those skilled in the art, upon attaining an understanding of the foregoing, may readily produce alterations to, variations of, and equivalents to such aspects. Numerous specific details are set forth herein to provide a thorough understanding of the claimed subject matter. However, those skilled in the art will understand that the claimed subject matter may be practiced without these specific details. In other instances, methods, apparatuses, or systems that would be known by one of ordinary skill have not been described in detail so as not to obscure claimed subject matter. Accordingly, the present disclosure has been presented for purposes of example rather than limitation, and does not preclude the inclusion of such modifications, variations, and/or additions to the present subject matter as would be readily apparent to one of ordinary skill in the art.

Unless specifically stated otherwise, it is appreciated that throughout this specification discussions utilizing terms such as “processing,” “computing,” “calculating,” “determining,” and “identifying” or the like refer to actions or processes of a computing device, such as one or more computers or a similar electronic computing device or devices, that manipulate or transform data represented as physical electronic or magnetic quantities within memories, registers, or other information storage devices, transmission devices, or display devices of the computing platform. The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or values beyond those recited. Headings, lists, and numbering included herein are for ease of explanation only and are not meant to be limiting.

Aspects of the methods disclosed herein may be performed in the operation of such computing devices. The system or systems discussed herein are not limited to any particular hardware architecture or configuration. A computing device can include any suitable arrangement of components that provide a result conditioned on one or more inputs. Suitable computing devices include multi-purpose microprocessor-based computer systems accessing stored software that programs or configures the computing system from a general-purpose computing apparatus to a specialized computing apparatus implementing one or more aspects of the present subject matter. Any suitable programming, scripting, or other type of language or combinations of languages may be used to implement the teachings contained herein in software to be used in programming or configuring a computing device. The order of the blocks presented in the examples above can be varied—for example, blocks can be re-ordered, combined, and/or broken into sub-blocks. Certain blocks or processes can be performed in parallel.

In some instances, one or more components may be referred to herein as “configured to,” “configurable to,” “operable/operative to,” “adapted/adaptable,” “able to,” “conformable/conformed to,” etc. Those skilled in the art will recognize that such terms (e.g., “configured to”) can generally encompass active-state components and/or inactive-state components and/or standby-state components, unless context requires otherwise.

As used herein, the term “based on” can be used synonymously with “based, at least in part, on” and “based at least partly on.”

As used herein, the terms “comprises/comprising/comprised” and “includes/including/included,” and their equivalents, can be used interchangeably. An apparatus, system, or method that “comprises A, B, and C” includes A, B, and C, but also can include other components (e.g., D) as well. That is, the apparatus, system, or method is not limited to components A, B, and C.

Although the subject matter has been described in language specific to structural features and/or methodological acts, it is to be understood that the subject matter defined in the appended claims is not necessarily limited to the specific features or acts described.

What is claimed is:

1. A system, comprising:
a handheld diagnostic tool comprising:
a housing defining optics and illumination for performing a manually guided vision exam of a patient; and
an attachment mechanism optically connected to the optical port; and
a device comprising:
a body defining a receiving surface on a first side of the housing;
a camera positioned within the channel; and
a display on a second side of the body, wherein the receiving surface is configured to releasably secure with the attachment mechanism to align the camera with the optical port.

2. The system of claim 1, wherein the attachment mechanism and receiving surface are configured to enable rotation of the device relative to the handheld diagnostic tool while maintaining alignment of the camera with the optical port.

3. The system of claim 1, wherein the device further comprises an orientation sensor configured to determine an orientation of the device.

4. The system of claim 3, wherein the orientation of the device comprises an orientation with respect to a gravitational reference.

5. The system of claim 3, wherein the orientation sensor comprises one or more sensors within the channel configured to detect an orientation of the eyepiece relative to the device.

6. The system of claim 3, wherein the display of the device is configured to display image data from the camera including an indication of the orientation of the device based at least in part on the orientation sensor.

7. The system of claim 1, wherein the receiving surface comprises a first portion adjacent a first edge of the device to receive the attachment mechanism and a locking portion at a center portion of the device connected with the first portion and configured to receive and rotationally secure the device with the handheld diagnostic tool.

8. The system of claim 1, wherein:
the handheld diagnostic tool comprises one or more detents adjacent the eyepiece; and
the device comprises one or more surfaces positioned radially around the camera to receive at least one of the one or more detents of the handheld diagnostic tool and frictionally resist movement of the device relative to the handheld diagnostic tool.

9. The system of claim 1, wherein the device further comprises a lock to selectively prevent removal of the device from the attachment mechanism.

10. The system of claim 1, wherein the device is configured to detect an interface between the receiving surface and the attachment mechanism and, in response to detecting the interface, cause the display and camera to activate.

11. A display device for a handheld diagnostic tool, the display device comprising:
a body;
an interface adjacent a surface of the body comprising:
a receiving portion adjacent the edge of the display device, the receiving portion defining an opening having a first width to receive an eyepiece of the handheld diagnostic tool;
a middle portion positioned between the edge of the display device and the center portion along the first direction, the middle portion having a second width at the surface of the body and a third width at a depth below the surface of the body, wherein the second width is less than the third width; and
a locking portion positioned adjacent the center portion of the display device comprising one or more features to interface with the eyepiece to frictionally retain the eyepiece in a position;
a camera positioned within the channel adjacent the center portion; and
a display communicably coupled to the camera and configured to display image data captured by the camera.

12. The display device of claim 11, further comprising an orientation sensor configured to determine an orientation of the display device relative to a gravitational reference frame, and wherein the display is configured to display orientation data associated with the orientation of the display.

13. The display device of claim 11, wherein the locking portion comprises an orientation sensor configured to detect an orientation of the handheld diagnostic tool relative to the display device.

14. The display device of claim 11, wherein the locking portion comprises a frictional interface configured to releasably rotationally secure the display device with the eyepiece.

15. The display device of claim 14, wherein the frictional interface comprises one or more detents positioned around the camera.

16. The display device of claim 15, wherein the one or more detents comprise four detents spaced evenly about the camera.

17. The display device of claim 11, wherein the locking portion comprises a rectangular profile configured to receive a portion of the eyepiece in four orientations and resists rotation of the display device when the eyepiece is coupled with the locking portion.

18. The display device of claim 11, wherein the locking portion comprises a round profile configured to enable the display device to rotate with respect to the eyepiece while the eyepiece is coupled with the locking portion.

19. The display device of claim 11, further comprising one or more processors and one or more computer-readable media having instructions stored thereon that, when executed by the one or more processors, perform operations comprising:
receiving image data from the camera;
determining an orientation of the image data with respect to a reference frame of the patient by processing the image data using one or more algorithms; and
causing an indication of the orientation to visible on the image data or display of the display device.

20. A device, comprising:
a body defining a channel on a first side;
a camera positioned within the channel; and
a display on a second side of the body, wherein the channel is configured to releasably secure with an eyepiece of a handheld diagnostic tool to align the camera with an optical port of the handheld diagnostic tool and enable rotation of the device relative to the handheld diagnostic tool while maintaining alignment of the camera with the optical port.

21. The device of claim 20, further comprising an orientation sensor configured to determine an orientation of the device relative to a gravitational reference frame, and wherein the display is configured to display orientation data associated with the orientation of the device.

22. The device of claim 20, wherein the channel comprises a first end adjacent an edge of the device and a second end adjacent a center portion of the device, wherein the second end comprises a frictional interface to releasably rotationally secure the device with the eyepiece.

23. The device of claim 22, wherein the frictional interface comprises one or more detents positioned around the camera.

24. The device of claim 22, wherein the channel further comprises an orientation sensor positioned adjacent the second end and configured to detect an orientation of the handheld diagnostic tool relative to the device.
